(19) United States
(12) Patent Application Publication
(10) Pub. No.: US 2023/0160849 A1
(43) Pub. Date: May 25, 2023

(54) Engineered DNA for Molecular Electronics

(71) Applicant: Universal Sequencing Technology Corporation, Canton, MA (US)

(72) Inventors: Peiming Zhang, Gilbert, AZ (US); Predrag S. Krstic, Port Jefferson Station, NY (US); Ming Lei, Sharon, MA (US)

(73) Assignee: Universal Sequencing Technology Corporation, Canton, MA (US)

(21) Appl. No.: 17/777,877

(22) PCT Filed: Nov. 20, 2020
(86) PCT No.: PCT/US2020/061665

§ 371 Date: May 18, 2022

Related U.S. Application Data
(60) Provisional application No. 62/938,084, filed on Nov. 20, 2019.

(51) Int. Cl. G01N 27/414; C12Q 1/6869; G01N 27/327

(52) U.S. Cl. CPC G01N 27/4146; C12Q 1/6869; G01N 27/3278; G01N 27/4145

(57) ABSTRACT

The present invention is related to engineered nucleic acid bases for use in molecular electronics, such as nanosensors, molecular-scale transistors, FET devices, molecular motors, logic and memory devices, and nanogap electronic measuring devices for the identification and/or sequencing of biopolymers.

This application claims priority to U.S. Provisional Application Ser. No. 62/938,084 filed Nov. 20, 2019, the entire disclosures of which are hereby incorporated herein by reference.

### FIELD

The present invention is related to engineered nucleic acid bases for use in molecular electronics, such as nanosensors, molecular-scale transistors, FET devices, molecular motors, logic and memory devices, nanogap electronic measuring devices for the identification and/or sequencing of biopolymers, etc.

### BACKGROUND

Next-generation lithography (NGL) technologies, such as extreme ultraviolet (EVL), allow for a sub-10 nm process with high volume manufacturing.¹ With EVL ready for the 7 nm node, it makes the process cheaper and faster than the state-of-the-art 193-nm immersion lithography, The 5 nm node process is also feasible with NGL. Conventional microchip fabrication is energy and resource-intensive. Thus, the discovery of any new manufacturing approache that reduce these expenditures would be highly beneficial to the semiconductor industry.

The mentioned above developments in semiconductor industries pave the way for the ‘bottom-up’ assembly of sub-10 nm electronic components, such as transistors, from a single organic molecule.² DNA is one of the most attractive molecules for single-molecule electronics due to its uniform one-dimensional structure (˜2 nm diameter), programmable self-assembly through the Watson-Crick base-pairing rule (G base pairs with C and A with T), and a tunable length ranging from nanometer to micrometers with angstrom accuracy. Therefore, DNA has been studied as an ideal nanomaterial for building molecular electronics. However, the early measurements on charge transport in DNA showed that DNA acted as an insulator,³⁻⁶ a semiconductor,^{7,8} or a metal-like conductor.^{9,10} These contradicted observations may be caused by the measured samples (the structure, sequence, length of DNA, etc.), measurement environments, and methods,¹¹ knowing that the DNA structure is polymorphous, which changes with its surroundings. The development in the single molecule technology in the past fifteen years has facilitated single molecule conductivity measurement. For example, the single molecule break junction technique allows one to measure the conductivity of a single molecule repeatedly in an aqueous solution. A DNA duplex of TG₈C₈A has measured conductance of ˜72 nS under bias between 30 and 50 mV.¹² There is a consensus that short DNA is a one-dimensional semiconductor, and DNA is insulating at length scales longer than 40 nm^{6,13}. The conductance of DNA can be electrochemically gated¹⁴ and rectified by an intercalator.¹⁵ Intrinsically, a T-A base pair is less conductive than a G-C base pair in a DNA molecule, and mismatched base pairs also change the conductivity of DNA¹⁶. The conductivity of DNA exponentially reduces with its length of AT base pairs and decreases by 1/L with its length of G-C base pairs length.¹⁷ Thus, the AT base pair plays a barrier in electron transport through DNA when the conductivity is measured using noble metal electrodes, such as gold and platinum. These metal electrodes have their work functions closer to the HOMO energies than to the LUMO energies of nucleobases, acting as anodes for the hole injection.¹⁸ The base G has the lowest ionization potential among those naturally occurring nucleobases, and it is a definite strong hole acceptor.¹⁸ In the hopping model, G is a hopping site for the DNA conduction.^{18,20} Note that this dynamical disorder may be beneficial for hole transfer. It helps a charge carrier overcome the barrier formed by the electrostatic interactions between the propagating hole and the hole donor's anion.²¹

The contact of DNA to electrodes also affects its measured conductance significantly.²² Wagner and coworkers have used fullerenes (060) as anchors to connect DNA to gold electrodes.²³ They observed a long-range charge transport over more than 20 nm in a DNA molecule comprising 66.7% of GC base pairs with current intensities in the nano ampere range under bias between 0 to 1 V. However, the fullerene was conjugated to DNA through a 06 alkyl chain, which presents a high tunneling barrier with a decay constant (3) of 1.0 per methylene group.²⁴

In general, DNA is a macromolecule consisting of four deoxyribonucleosides, deoxyadenosine (dA), deoxycytidine (dC), and deoxyguanosine (dG), and thymidine (T), which are linked together via phosphodiester bonds. It can be synthesized chemically or enzymatically, which allows for engineering DNA with a variety of modifications. Although homogeneous sequences containing only guanine-cytosine (G:C) base pairs exhibit relatively high hole mobility for charge transport, their synthesis with long chains and high purity is difficult. Besides, the GC rich DNA is prone to form undesired secondary and even quadruple structures.

### BRIEF DESCRIPTION OF THE DRAWINGS

FIG. 1 illustrates the general structure of engineered DNA in this invention.

FIG. 2 shows HOMO and LUMO structures of 5-propenyl deoxyuridine (101) and its natural counterpart, thymidine, determined by DFT calculation.

FIG. 3 shows HOMO and LUMO structures of 8-(3-mercaptopropynyl)-deoxyguanosine (201) and its parent nucleoside deoxyguanosine determined by DFT calculation.

FIG. 4 shows HOMO and LUMO structures of 7-propenyl-7-deaza-deoxyadenosine (301) and its parent nucleoside deoxyguanosine determined by DFT calculation.

FIG. 5 shows hydrogen bonding patterns, as well as their HOMO and LUMO structures, of base pairs between canonical DNA bases and modified bases.

FIG. 6 shows (a) Configuration of DNA duplex-1 connected to metal electrodes and its transmission spectra (listed in Table 5), (b) Configuration of DNA duplex-2 connected to metal electrodes and its transmission spectra (listed in Table 5).

FIG. 7 shows I-V curves of DNA Duplex-1 and Duplex-2 and their differential conductance spectra.

FIG. 8 shows the Configuration of DNA Duplex-3 (listed in Table 5) connected to metal electrodes (a), its transmission spectra (b), I-V curves of both DNA Duplex-1 and Duplex-3 (c), and their differential conductance spectra (d).

FIG. 9 shows the Configuration of DNA Duplex-4 (listed in Table 5) connected to metal electrodes (a), its transmission spectra (b), I-V curves of both DNA Duplex-1 and Duplex-4 (c), and their differential conductance spectra (d).

FIG. 10 shows the Configuration of DNA Duplex-5 (listed in Table 5) connected to metal electrodes (a), its transmission spectra (b), I-V curves of both DNA Duplex-1 and Duplex-2 as well as Duplex-5 (c), and their differential conductance spectra (d).

FIG. 11 shows the Configuration of DNA Duplex-6 connected to metal electrodes (a), its transmission spectra (b), I-V curves of both DNA Duplex-6 and Duplex-2 (c), and their differential conductance spectra (d).

FIG. 12 shows the Configuration of DNA Duplex-7 connected to metal electrodes (a), its transmission spectra (b), I-V curves of both DNA Duplex-7 and Duplex-5 (c), and their differential conductance spectra (d).

### SUMMARY OF THE INVENTION

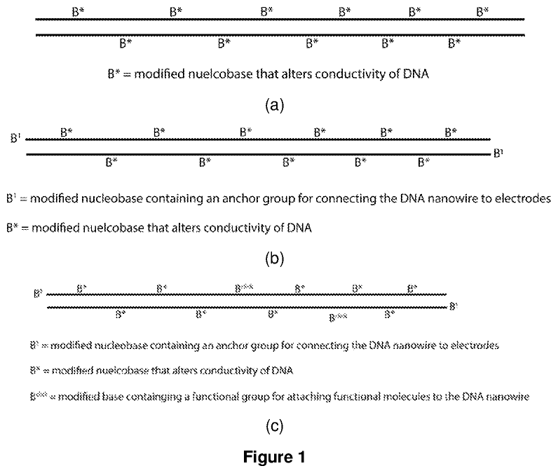

This invention provides DNA engineered with modified nucleobases, as shown in FIG. 1a, which appear on either strand or strands of a DNA duplex. The modified base improves the conductance of DNA and retains its base-pairing specificity. The engineered DNA can be used as a building element of a molecular electronic circuit, a nanosensor, and other nanoscale electronic devices. The engineered DNA comprises molecular anchors (B1 in FIG. 1b) at its ends for attachment to electrodes to bridge the nanogap and/or functional groups (B^{click} in FIG. 1c) for conjugation with other chemo and biological molecules in use for the chemical and biological sensing. These modifications can be readily incorporated into DNA by chemical or enzymatical synthesis.

The engineered DNA can be used in a nanogap electronic measuring device for the identification and/or sequencing of biopolymers, such as but not limited to the devices disclosed in patent applications, US20170044605A1, US20180305727A1 and also in provisional patent applications, U.S. 62/794,096, U.S. 62/812,736, U.S. 62/833,870, U.S. 62/890,251, U.S. 62/861,675, and U.S. 62/853,119, including nanostructure or nanogap devices to identify and/or sequence DNAs, RNAs, proteins, polypeptides, oligonucleotides, polysaccharides, and their analogies, etc., either natural, synthesized, or modified. The chemical or sensing probes in those devices disclosed in the above patent applications include but are not limited to nucleic acid probes, molecular tweezers, enzymes, receptors, ligands, antigens, and antibodies, either native, mutated, expressed, or synthesized, and a combination thereof. The enzymes include but are limited to DNA polymerase, RNA polymerase, DNA helicase, DNA ligase, DNA exonuclease, reverse transcriptase, RNA primase, ribosome, sucrase, lactase, etc., either natural, mutated, or synthesized.

### DETAILED DESCRIPTION OF THE INVENTION

The invention first provides modified nucleosides and their phosphoramidites, 5-alkenyl-2′-deoxyuridines, for the engineering of DNA. As shown in Scheme 1, these compounds are synthesized by following a procedure published in the literature,²⁵ where R is an alkyl group, such as methyl, ethyl, propyl, iso-propyl, butyl, iso-butyl, tert-butyl, cyclopropyl, cyclohexyl, but not limited to them, or halogenated alkyl such as trifluoromethyl, and also an aromatic ring, such as benzene, five-membered heterocycles, and their derivatives.

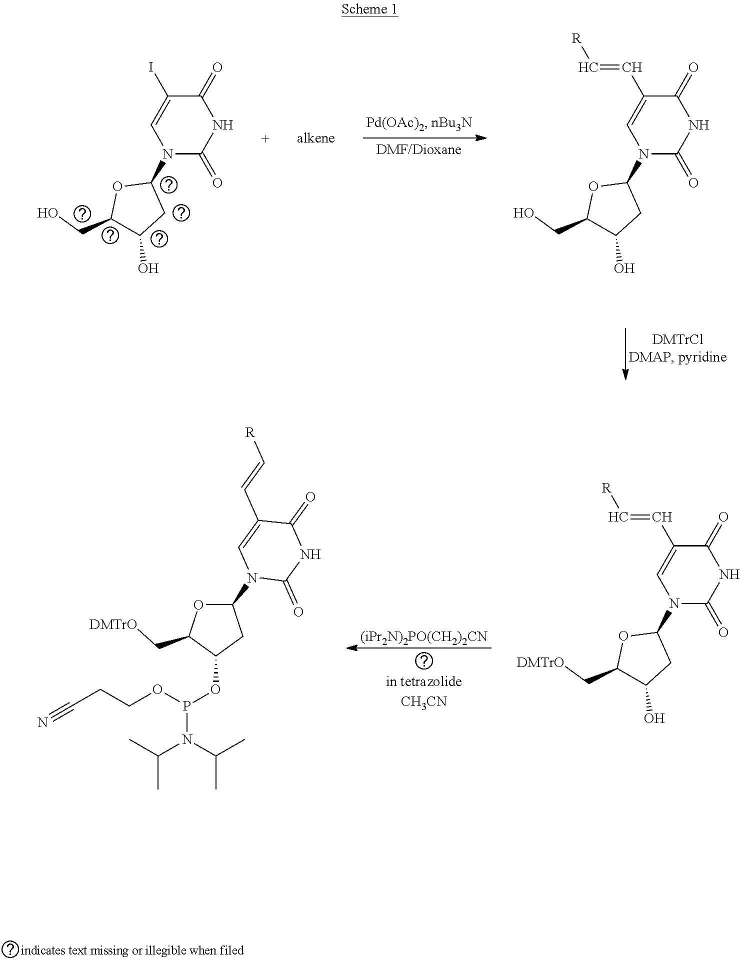

In some embodiments of this invention, DNA is engineered by replacing thymidine with modified uridine 101 (denoted by U^{m}) that has a chemical structure as shown below:

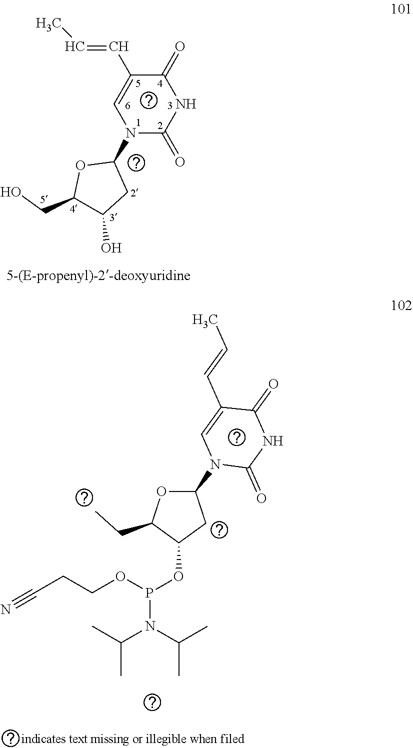 [SMILES: C/C=C/c1cn([C@H]2C[C@H](O)[C@@H](CO)O2)c(=O)[nH]c1=O ; C/C=C/c1cn([C@H]2C[C@H](OP(OCCC#N)N(C(C)C)C(C)C)[C@@H](CC)O2)c(=O)[nH]c1=O]

Specifically, nucleoside 101 is a derivative of 2′-deoxyuridine with a propenyl group attached to its carbon 5, in which the double bond of the propenyl group has an E configuration. Nucleoside 101 is converted to phosphoramidite 102 for its incorporation into DNA by chemical synthesis (Scheme 2).

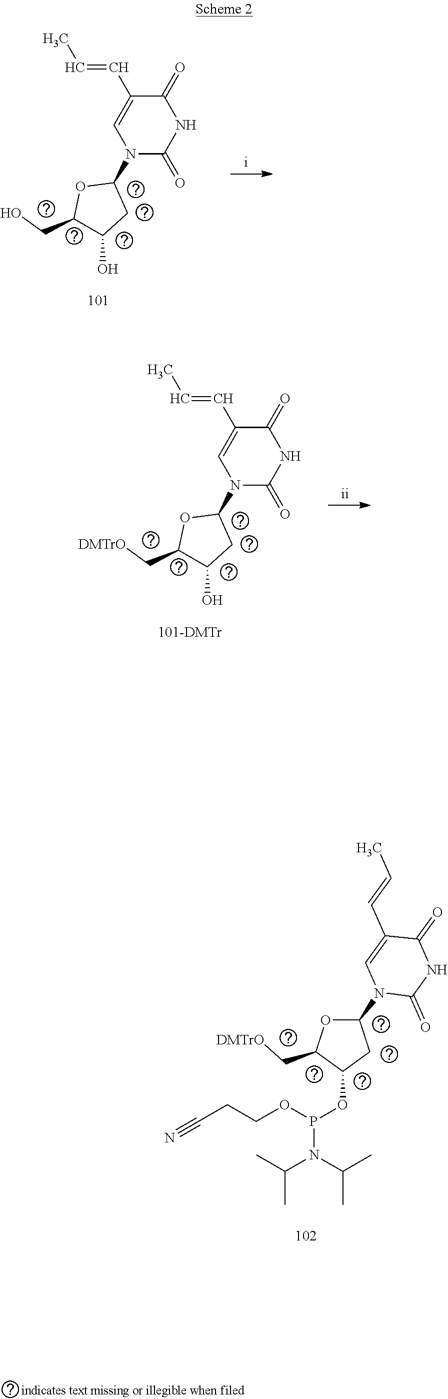

Procedure (i): Nucleoside 101 is dried by repeated co-evaporation with dry pyridine and dissolved in anhydrous pyridine, followed by the addition of 4,4′-Dimethoxytrityl chloride, 4-dimethylaminopyridine, and freshly distilled triethylamine. The solution was stirred under a nitrogen atmosphere, monitored by an analysis of the crude reaction mixture by TLC (CHCl3/ethanol 10:1) until the absence of the free nucleoside. The reaction mixture is quenched by the dropwise addition of water and extracted (3×40 ml) with ethyl ether. The organic layers were combined, dried over Na₂SO₄, filtered, and evaporated under reduced pressure to an oily residue. The product 101-DMTr is separated from the residue using column chromatography on silica gel.

Procedure (ii): Nucleoside 101-DMTr is dried by repeated co-evaporation with acetonitrile three times in vacuo and dissolved in anhydrous CH₂Cl₂. To this solution was added diisopropylammonium tetrazolide along with 2-cyanoethyl N,N,N′,N′-tetraisopropylphosphorodiamidite. The solution was allowed to stand under nitrogen at ambient temperature with occasional gentle swirling, monitored by TLC until the complete absence of starting material. The reaction mixture is cooled to 0° C. and quenched by the dropwise addition of methanol/0.5% TEA. The solution is evaporated under reduced pressure to an oily residue. The residue is dissolved in CH₂Cl₂ and washed with a saturated solution of NaHCO₃ two times, dried over Na₂SO₄, and evaporated under reduced pressure to an oily residue. The product 102 is purified by chromatography eluting with hexane/ethyl acetate/TEA (1%).

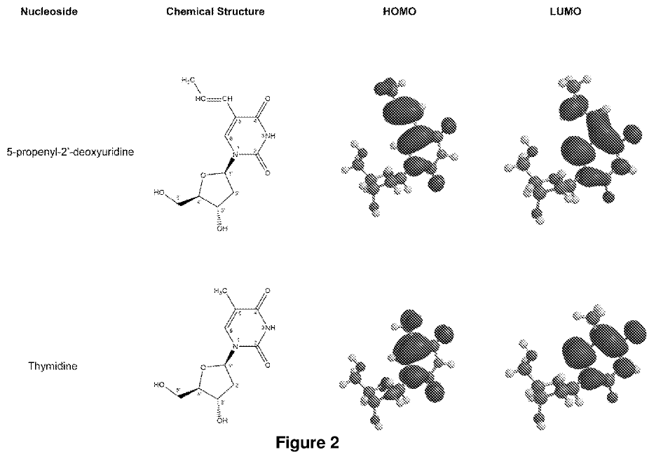

The calculation by density functional theory (DFT) indicates that the modified deoxyuridine 101 (U^{m}) has a higher HOMO and lower LUMO energies with 0 eV referenced as the highest point, resulting in a smaller energy gap between HOMO and LUMO, compared to its parent nucleoside deoxythymidine (Table 1). FIG. 2 shows that both HOMO and LUMO of these nucleosides are situated in their nucleobases. The modified base Um has a higher HOMO and lower LUMO than the natural base T.

TABLE 1
Structural Properties of nucleoside 101 calculated by DFT (B3LYP/6-311 + G(2df, 2pl)

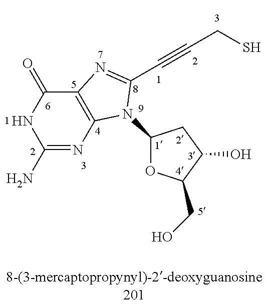 [SMILES: Nc1nc2c(nc(C#CCS)n2[C@H]2C[C@H](O)[C@@H](CO)O2)c(=O)[nH]1]

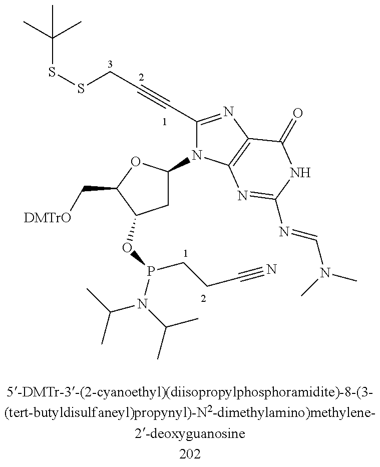

| | Molecular Orbital Energy | | | |
|---|---|---|---|---|
| | HOMO | LUMO | Energy Gap | |
| Nucleoside | (eV) | (eV) | (ev) | Dipole Moment (debye) |
| Thymidine (T) | −6.58 | −1.23 | 5.35 | 7.16 |
| 5-propenyldeoxythymidine (101) | −6.07 | −1.52 | 4.55 | 7.31 |

In one embodiment, U^{m} is chemically incorporated into DNA by an automated DNA synthesizer. One exemplary sequence is 5′-CGCGU^{m}CGCG²⁰¹, which also includes a modified guanosine 201 (denoted by G²⁰¹ or ²⁰¹G) at its 3′-end for its attachment to metal electrodes. The modified G²⁰¹ can be incorporated into DNA through its phosphoramidite derivative (202), which is synthesized following a prior art method²⁶.

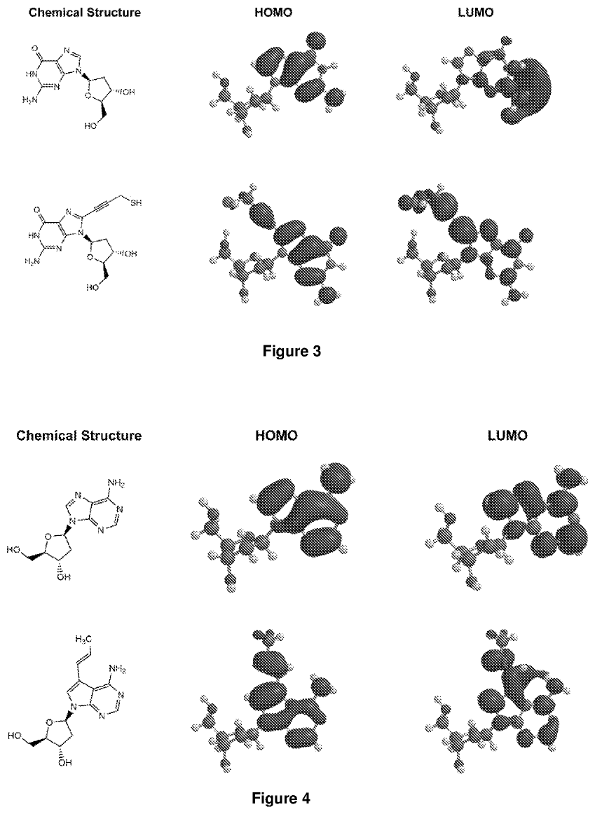

In one embodiment of this invention, HOMOs and LUMOs of nucleoside 201 and its parent nucleoside deoxyguanosine are determined by DFT calculation, listed in Table 2. FIG. 3 shows the HOMO and LUMO of these nucleosides, which are situated in their respective nucleobases. Moreover, the modification does not change the HOMO energy level. Still, it lowers the LUMO energy level, which implies that the modified guanine should have the same efficiency as the native guanine or better for the hole injection from the electrodes.

TABLE 2
Structural Properties of nucleoside 201 calculated
by DFT (B3LYP/6-311 + G(2df, 2p))

| | Molecular Orbital Energy | | | |
|---|---|---|---|---|
| | HOMO | LUMO | Energy Gap | Dipole Moment |
| Nucleoside | (eV) | (eV) | (ev) | (debye) |
| Deoxyguanosine | −5.84 | −0.60 | 5.32 | 6.88 |
| 8-(3-mercaptopropynyl)- | −5.86 | −1.32 | 4.67 | 5.21 |
| deoxyguanosine (201) | | | | |

In some embodiments of this invention, DNA is engineered by replacing deoxyadenosine with modified deoxyadenosine 301 (denoted by A^{m}) that has a chemical structure as shown below. As shown in Scheme 3, nucleoside 301 is synthesized first by running the Suzuki coupling reaction (Reaction i),²⁷ and then protecting the amino group of 301 with a benzoyl group (Reaction ii),²⁸ which is, in turn, converted into its corresponding phosphoramidite in the same way as described in Section [0020] (Reaction iii). The methyl (CH₃) group in the structure can be substituted with another alkyl group, such as ethyl, propyl, iso-propyl, butyl, iso-butyl, tert-butyl, cyclopropyl, cyclohexyl, but not limited to them, or halogenated alkyl such as trifluoromethyl, or with an aromatic ring, such as benzene, five-membered heterocycles,

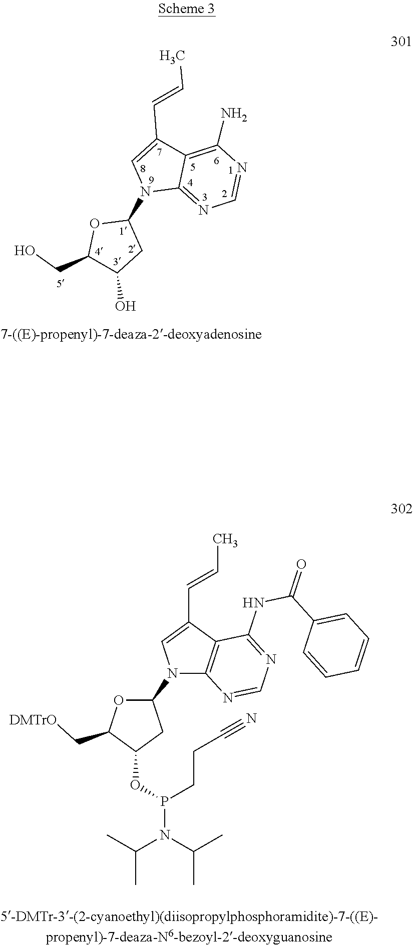

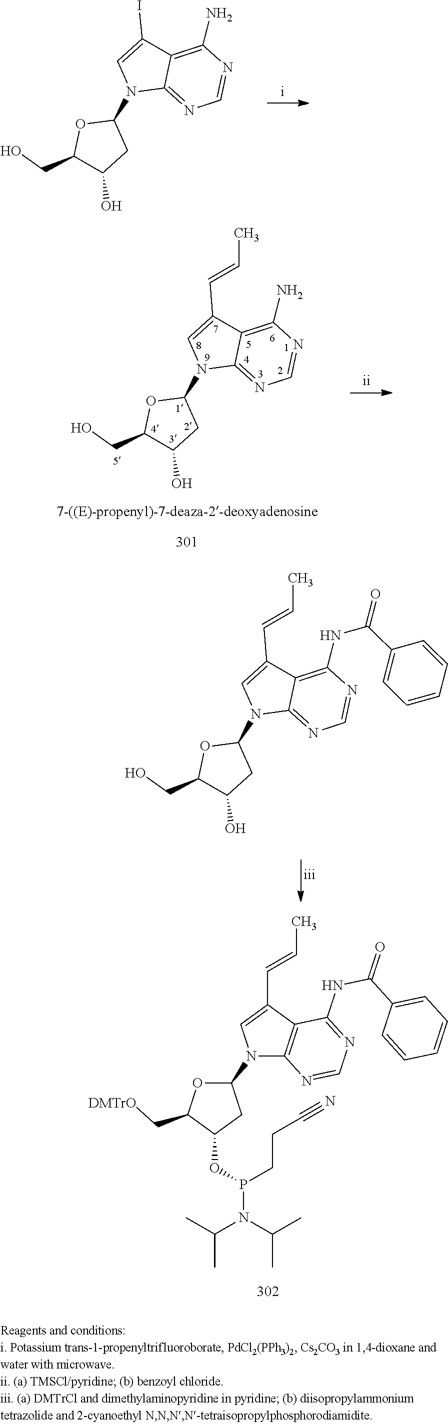

and their derivatives, but not limited to them.

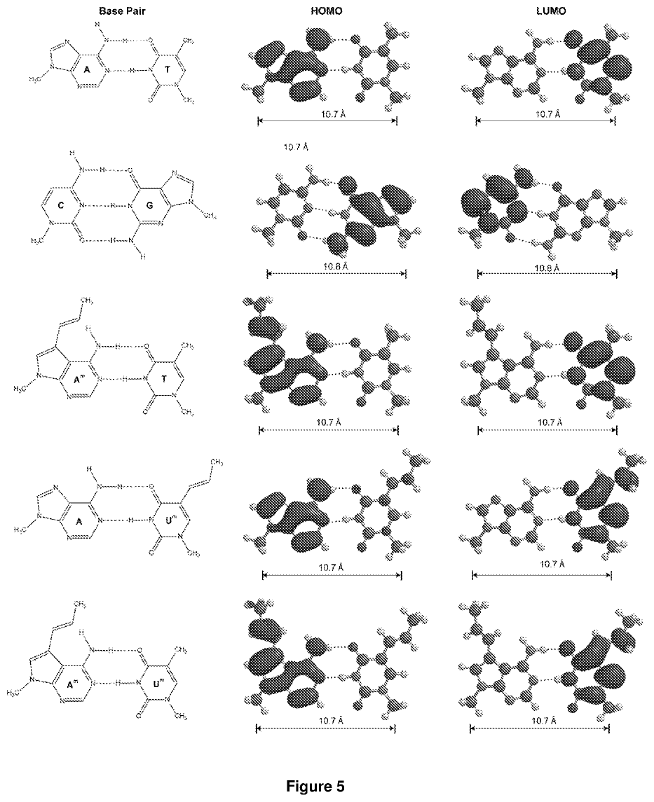

DFT calculation indicates that the modified deoxyadenosine 301 (A^{m}) has a higher HOMO and lower LUMO energies, resulting in a smaller energy gap between its HOMO and LUMO in comparison to the naturally occurring parent deoxyadenosine (Table 2). FIG. 4 shows the HOMOs and LUMOs of these nucleosides, which are situated in their respective nucleobases.

TABLE 3
Structural Properties of nucleoside 301 calculate
by DFT (B3LYP/6-311 + G(2df, 2p))

| | Molecular Orbital Energy | | | |
|---|---|---|---|---|
| | HOMO | LUMO | Energy Gap | Dipole Moment |
| Nucleoside | (eV) | (eV) | (ev) | (debye) |
| Deoxyadenosine | −6.09 | −0.74 | 5.32 | 5.09 |
| 7-propenyl-7- | −5.50 | −0.83 | 4.67 | 5.48 |
| deaza-deoxyadenosine | | | | |

Table 4 lists the molecular orbital energy of hydrogen bonding base pairs, determined by DFT calculation, which includes the naturally occurring Watson-Crick base pairs as well as modified A (A^{m}) base pairing with T and A with modified U (U^{m}). Since the HOMOs and LUMOs of the said nucleosides are situated in their respective nucleobases, their sugar rings are replaced by methyl groups (FIG. 5) to reduce the CPU time of DFT calculation for these base pairs. As shown in FIG. 5, all of the HOMOs are located in the purine rings, and LUMOs in the pyrimidine rings for these said base pairs. The energy levels of their HOMOs and LUMOs are listed in Table 4. Compared to the A:T base pair, A^{m}:T has a higher HOMO energy level and a comparable LUMO level; in contrast, A:U^{m} has a lower LUMO energy and a similar HOMO level. In contrast, A^{m}:U^{m} has both a higher HOMO and a lower LUMO level than A:T base pair. Compared to the C:G base pair, A^{m}:T has a higher LUMO energy level and a comparable LUMO level; A:U^{m} has a lower HOMO and a lower LUMO level; A^{m}:U^{m} has a lower LUMO and a similar HOMO level. Besides, the dipole moments of these base pairs are increased, compared to the native A:T base pair, which may increase the base stacking interactions between the neighbor base pairs.

TABLE 4
Structural Properties of base pairs calculated
by DFT (B3LYP/6-311 + G(2df, 2p))

| | Molecular Orbital Energy | | |
|---|---|---|---|
| | HOMO | LUMO | Dipole Moment |
| Base Pairing | (eV) | (eV) | (debye) |
| A:T | −6.03 | −1.34 | 1.74 |
| C:G | −5.38 | −1.50 | 6.05 |
| A:U^{m} | −6.03 | −1.64 | 1.90 |
| A^{m}:T | −5.44 | −1.33 | 2.09 |
| A^{m}:U^{m} | −5.44 | −1.62 | 2.10 |

In this invention, the conductances of DNA are determined using Non-Equilibrium Green's Functions formalism (NEGF), which is a theoretical framework for modeling electron transport through nanoscale devices²⁹⁻³³. First, the transmission function T(E) is computed, which describes the probability of the charge transport at energy E from the left electrode to the right electrode by propagating through the scattering region. With the transmission function, electric currents are calculated for given electrical bias voltages applied between the electrodes, using Landauer-Buttiker formalism (S. Datta, Electronic Transport in Mesoscopic Systems, Cambridge University Press, Cambridge, 1995)

$$I ( V ) = \frac{2 e}{h} {\int }_{- ∞}^{∞} T ( E ) [ f ( E - {μ}_{S} ) - f ( E - {μ}_{D} ) ) dE$$

Where f(E) is the Fermi-Dirac distribution function for a given electronic temperature, and chemical potentials μ_{S}, μ_{D} are E_{f}+V and E_{f}, respectively. E_{f} is the Fermi level of the respective electrode (usually equal for source and drain).

In some embodiments, DNA duplexes comprise a palindromic sequence 5′-CGCG-X-CGCG with a base pair X in its middle (Table 5). For Duplex-1 and 2, X is canonical A:T and C:G base pairs, respectively. In the rest of the duplexes, X is A^{m}:T, A^{m}:U^{m}, or U^{m}:A. These modified bases can form the canonical Watson-Crich hydrogen-bonded base pairs with naturally occurring bases and between themselves, as shown in FIG. 5. In these base pairs, their HOMOs are mainly situated at the purine bases and LUMOs at the pyrimidine bases. Each of these duplexes carries modified Gs (²⁰¹G in this case) at 3′-ends for its attachment to metal electrodes (gold in this case). For current flow, the hole is injected into the guanine via the electrode to which it is connected; then, the charge is transported to another electrode through the DNA wire.

TABLE 5
DNA duplexes comprising sequences containing
modified single T^{m} and A^{m}

| Entry | DNA sequences |
|---|---|
| Duplex-1 | 5′-CGCG-A-CGCG²⁰¹ |
| | ²⁰¹GCGC-T-GCGC-5′ |
| Duplex-2 | 5′-CGCG-C-CGCG²⁰¹ |
| | ²⁰¹GCGC-G-GCGC-5′ |
| Duplex-3 | 5′-CGCG-A^{m}-CGCG²⁰¹ |
| | ²⁰¹GCGC-T-GCGC-5′ |
| Duplex-4 | 5-CGCG-A^{m}-CGCG²⁰¹ |
| | ²⁰¹GCGC-U^{m}GCGC-5′ |
| Duplex-5 | 5′-CGCG-U^{m}CGCG²⁰¹ |
| | ²⁰¹GCGC-A-GCGC-5′ |

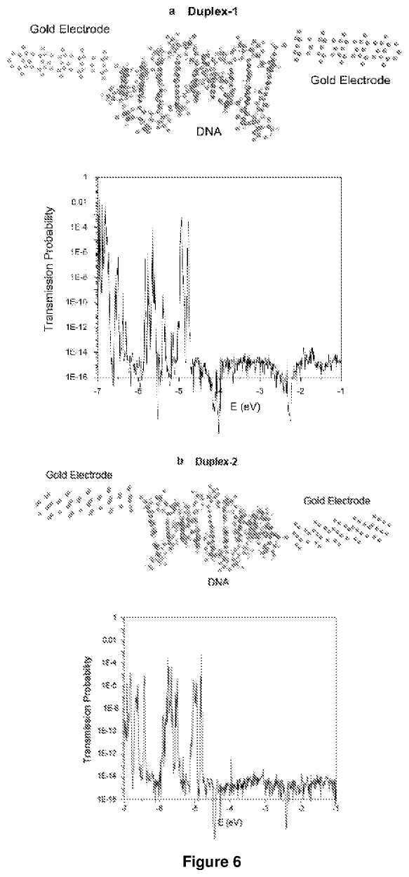

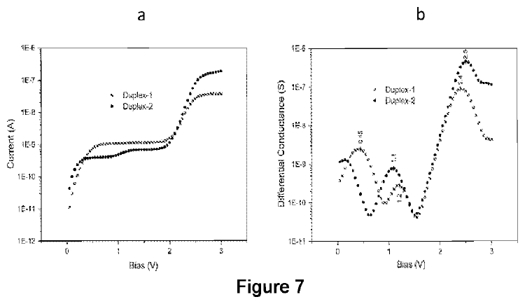

In one embodiment, both Duplex-1 and Duplex-2 are attached to two electrodes through the guanines at their ends, respectively, as shown in FIG. 6. The transmission spectra of electron transport through Duplex-1 and Duplex-2 were determined by the above-said computing. In turn, their conductances are derivated from the transmission spectrum by the above-said method as well. The I-V curves for these duplexes are generated in a range of 0 to 3 V, shown in FIG. 7a. First, both Duplex-1 and Duplex-2 solely comprise natural nucleobases, and the only difference between them is the base pair in the middle of their sequences. The results show that Duplex-2 is slightly more conductive than Duplex-1 in a bias close to zero (˜0 to 0.25 v), which is consistent with those reported in the literature because the AT base pair reduces the conductivity of DNA molecules. In bias in a range of 0.5 to 2.0 V, Duplex-2 becomes less conductive than Duplex-1. With further increase in the voltage bias, Duplex-2 becomes more conductive than Duplex-1 again. FIG. 7b shows differential conductance curves of Duplex-1 and Duplex-2. Their transitions are situated at different voltage biases, which reflects that they have a different local density of states (LDOS).

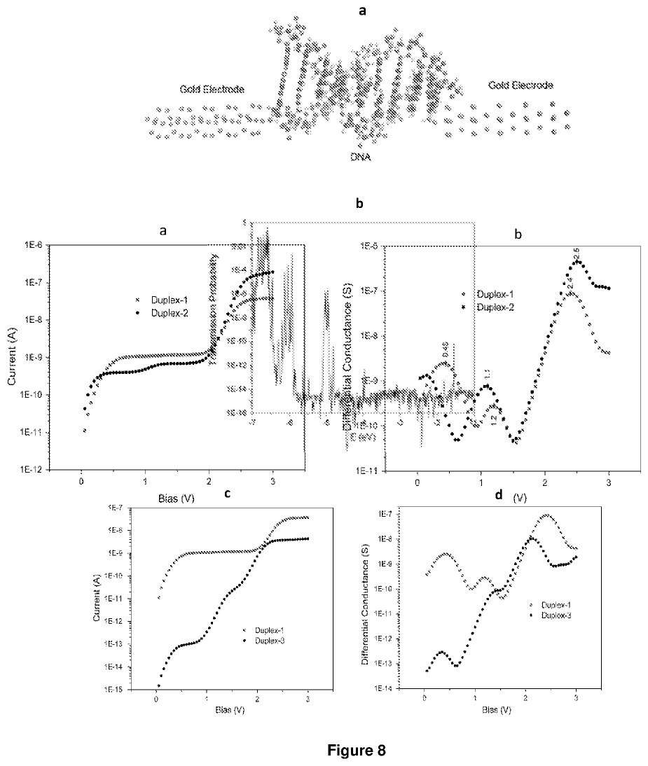

In another embodiment, the conductance of Duplex-3, in which A^{m} (301) replaces the nucleobase A of Duplex-1, was determined by the said method in Section [0027]. Duplex-3 is connected to the gold electrodes in the same way as Duplex-1 and Duplex-2 (FIG. 8a), with which its transmission spectrum is computed, shown in FIG. 8b. As shown in FIG. 8c, the modification on the nucleobase A reduces the conductivity of the DNA duplex significantly in the low bias range. With the increase of the voltage bias, the conductance of both Duplex-1 and Duplex-3 increases at a similar rate to reach their first plateaus. Shortly, Duplex-3 has its conductance increases at a rate same as the previous one to have its second plateau. In contrast, Duplex-1 keeps its conductance unchanged in a range of 1 to 2 V and then increases at a similar rate as the previous one to reach its second plateau. As a result, the conductance difference between these two DNA duplexes becomes much smaller at the higher biases than at the lower ones. These results are also reflected in their differential conductance (FIG. 8d), where Duplex-3 has a dynamic range much larger than Duplex-1.

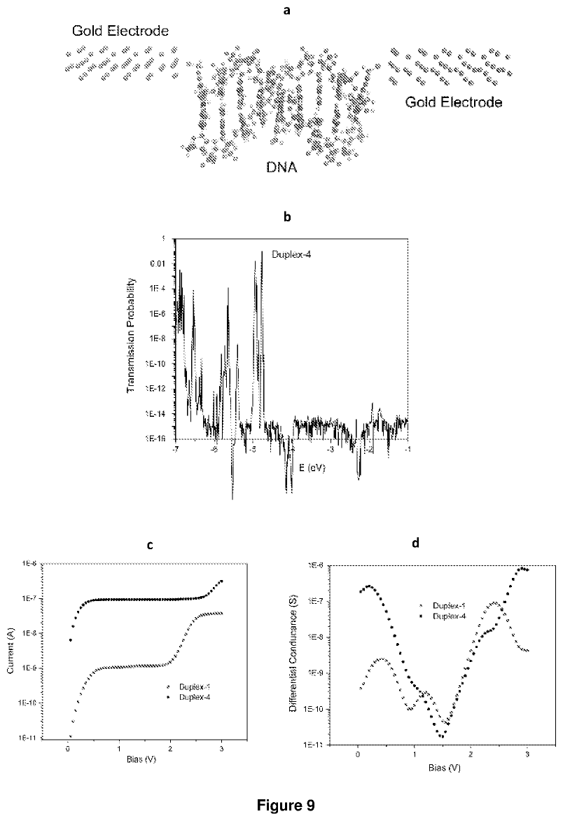

In one embodiment, the conductance of Duplex-4, in which A^{m}:U^{m} replaces the A:T base pair of Duplex-1, was determined by the said method in Section [0027]. Duplex-4 is connected to the gold electrodes in the same way as Duplex-1 (FIG. 9a). Based on the configuration, the transmission spectrum of Duplex-4 is computed, shown in FIG. 9b, and the I-V curve in FIG. 9c. For comparison, the I-V curve of Duplex-1 is also included in FIG. 9c. Overall, Duplex-4 is more conductive than Duplex-1. Particularly, Duplex-4 has a conductance of 5.8×10² times higher than Duplex-1 at the low bias. From their differential conductances (FIG. 9d), the conductance of both duplexes increase with voltage biases; however, Duplex-4 reaches a peak earlier than Duplex-1 does in the lower bias range. In the higher bias range, then, Duplex-1 reaches its high peak earlier than Duplex-4. These results show that the base pair modification increases the conductance of DNA.

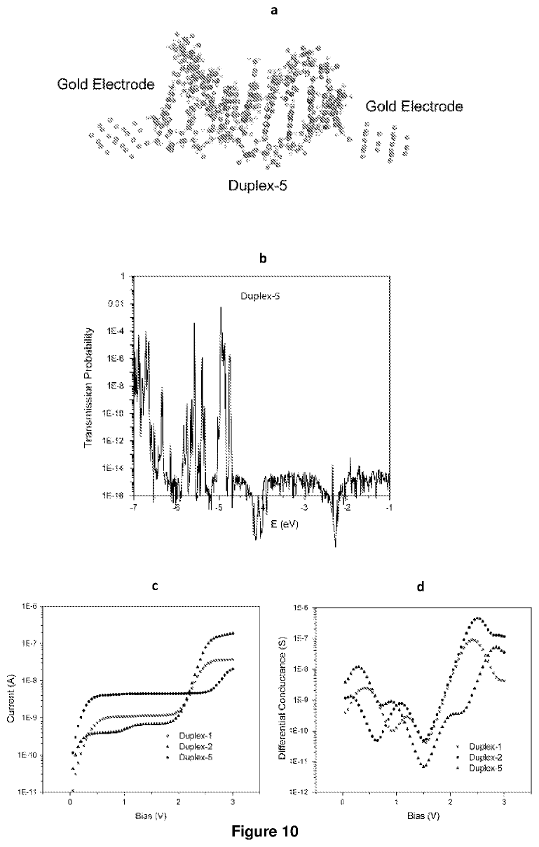

In one embodiment, the conductance of Duplex-5, in which the U^{m}:A base pair replaces the C:G base pair of Duplex-2, was determined by the said method in Section [0027]. Duplex-5 is connected to the gold electrodes in the same way as Duplex-2 (FIG. 10a). Based on the configuration, the transmission spectrum of Duplex-5 is computed, shown in FIG. 10b, and the I-V curve in FIG. 10c. For comparison, the I-V curves of Duplex-1 and Duplex-2 are also included in FIG. 10c. The data shows that the conductance of Duplex-5 can be one order of magnitude higher than that of Duplex-1 and four times higher than that of Duplex-2 in a bias range of 0 to 2 V, indicating that the U^{m}:A base pair increases the conductivity of DNA compared to the naturally occurring A:T and C:G base pairs. At the higher bias (>2 V), the naturally occurring base pairs become more conductive than the modified U^{m}:A base pair. Thus, the modified base can be used in the low voltage operation to increase the conductivity of DNA. Also, the conductance of Duplex-5 changes most at 0.3 V (FIG. 10d), at which it may provide higher sensitivity for sensing.

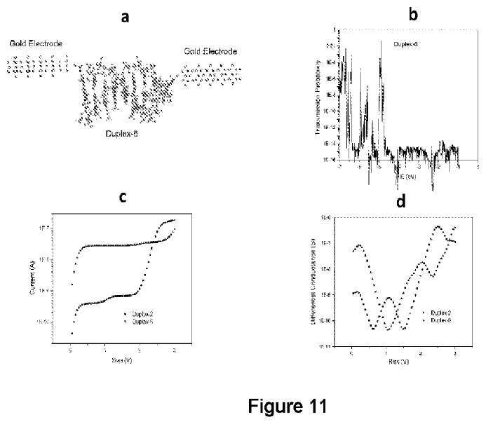

In one embodiment, the internal C:G base pairs of Duplex-2 have completely replaced with the A:U^{m} base pairs, which constitutes Duplex-6 with a form as below:
5′-C-A-U^{m}-A-U^{m}-A-U^{m}-A-G²⁰¹
²⁰¹G-U^{m}-A-U^{m}-A-U^{m}-A-U^{m}-C-5′
   It is connected to the gold electrodes in the same way as Duplex-2 (FIG. 11a). Based on the configuration, its conductance was computed by the said method described in Section [0027]. First, its transmission spectrum is computed, shown in FIG. 11b, and its I-V curve in FIG. 11c. For comparison, the I-V curve of Duplex-2 is also included in FIG. 11c. The data show that Duplex-6 is between 30 and 70 times more conductive than Duplex-2 in a bias range of 0 to 2 V. Compared to Duplex-5, in which one U^{m}:A base pair substitutes for the middle C:G base of Duplex-2, the multiple U^{m}:A substitution creates some degree of synergistic effect. However, Duplex-2 is more conductive than Duplex-6 at the high voltage biases (>2.0). Thus, the conductance of a DNA molecule can be changed by replacing G:C base pair with the modified U^{m}:A base pair. FIG. 11d shows that these two duplexes have different transition states, and their first transitions take place at a similar position (˜0.2 V). The U^{m}:A base pair interact with each other by forming two hydrogen bonds, whereas the G:C base pair by three hydrogen bonds. As a result, Duplex-6 should be more flexible than Duplex-2 and more sensitive to the external stimulation for sensing. However, Duplex-2 has more distinct transition states than Duplex-6, as shown in their differential conductance (FIG. 11 d).

In some embodiments, the invention also provides a modified guanosine 401 (denoted by G⁴⁰¹ or ⁴⁰¹G) to attach DNA to metal electrodes. In the same way as G^{m}, the modified G⁴⁰¹ can be incorporated into DNA through its phosphoramidite derivative with a disulfide form (402), which is synthesized following a prior art method. The disulfide can be reduced to thiol for the attachment to metal electrodes before use.

In one embodiment, Duplex-7 was synthesized by replacing G²⁰¹ of Duplex-5 with G⁴⁰¹, which form a duplex as shown below:

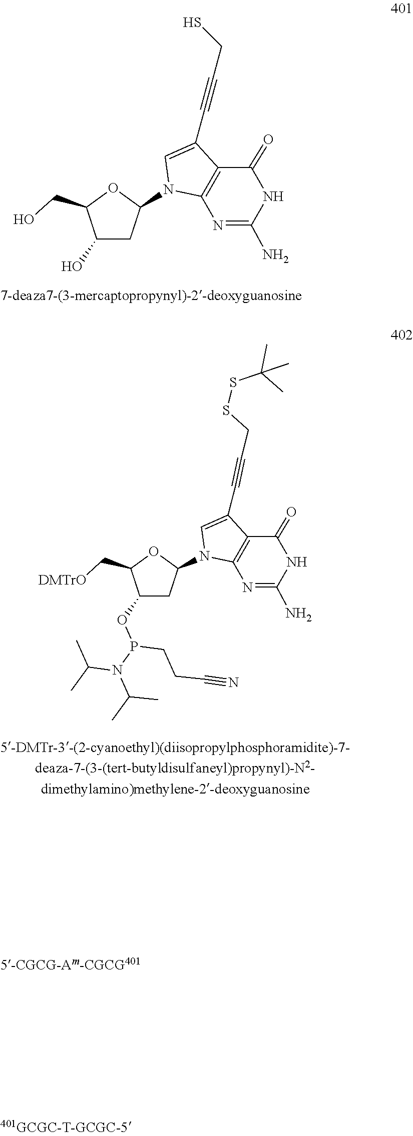

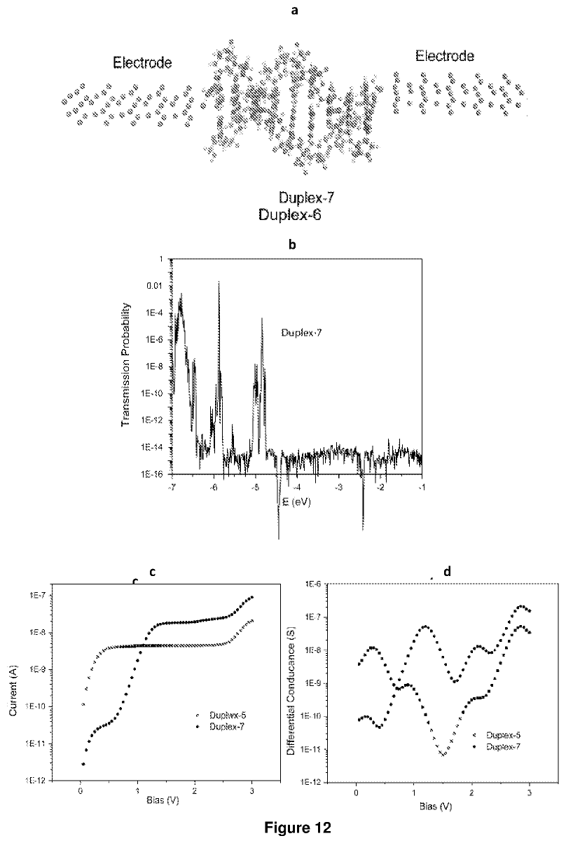

It is connected to the gold electrodes in the same way as Duplex-5 (FIG. 12a). Based on the configuration, its conductance was computed by the said method described in Section [0027]. First, its transmission spectrum is computed, shown in FIG. 12b, and its I-V curve in FIG. 12c. In the low voltage bias (0 to 1 V), Duplex-5 is more conductive as much as two orders of magnitude than Duplex-7. However, Duplex-7 is as much as five times more conductive than Duplex-5 in a high voltage bias. Besides, Duplex-7 has higher differential conductance than Duplex-5 when the voltage is higher than 0.7 V.

The invention provides 5-alkenyl-2′-deoxycytidines and their phosphoramidites for the engineering of DNA. These compounds are synthesized, as shown in Scheme 4, where R is an alkyl group, for example, methyl, ethyl, propyl, iso-propyl, butyl, iso-butyl, tert-butyl, cyclopropyl, cyclohexyl, but not limited to them, or halogenated alkyl such as trifluoromethyl. Also, R is an aromatic ring, such as benzene, five-membered heterocycles, and their derivatives.

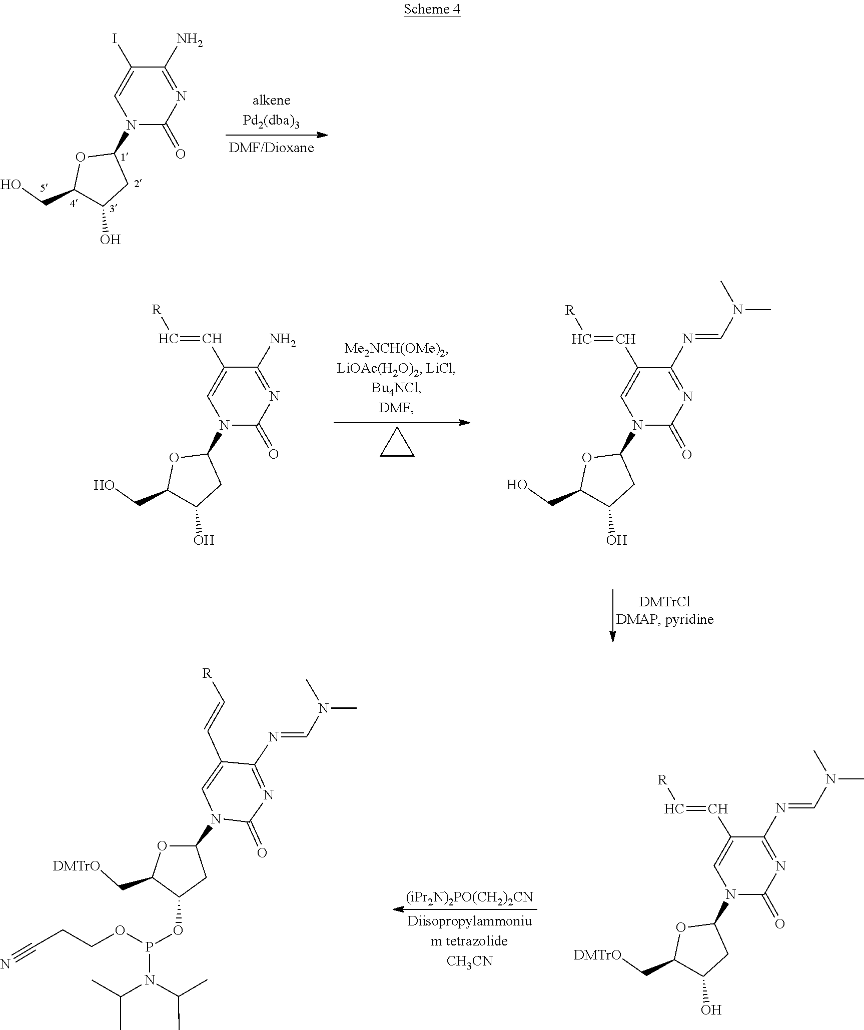

The invention provides 7-deaza-7-alkenyl-2′-deoxyguanosine to complement 5-alkenyl-2′-deoxycytidines for the base pairing in DNA as shown below, where R is methyl, ethyl, propyl, iso-propyl, butyl, iso-butyl, tert-butyl, cyclopropyl, cyclohexyl, but not limited to them, or halogenated alkyl such as trifluoromethyl. Also, R is an aromatic ring, such as benzene, five-membered heterocycles, and their derivatives. These compounds are synthesized following the method mentioned in Section [0024].

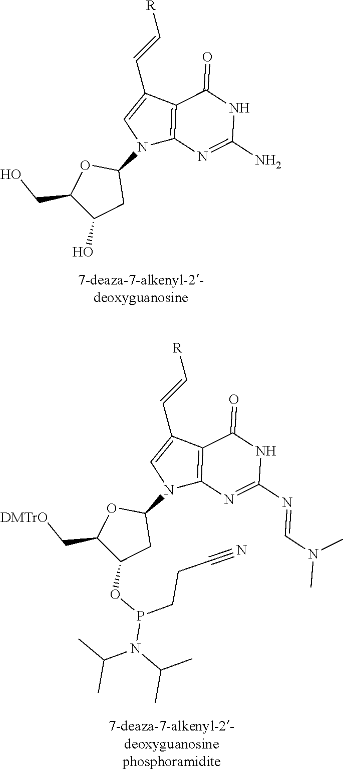

The invention provides nucleoside triphosphates, as shown below, where B is a modified nucleobase mentioned above, for incorporating the modifications into DNA enzymatically. The enzyme is a DNA polymerase that can extend a DNA chain with or without a template.

In some embodiments, the engineered DNA with one or more nucleobases

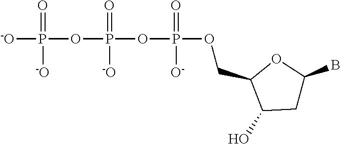

modified using the said methods or schemes discussed in this disclosure can be used in a nanogap electronic measuring device for the identification and/or sequencings of biopolymers, such as but not limited to the devices disclosed in patent applications, US20170044605A1, US20180305727A1 and also in provisional patent applications, U.S. 62/794,096, U.S. 62/812,736, U.S. 62/833,870, U.S. 62/890,251, U.S. 62/861,675, and U.S. 62/853,119. Specifically, it can be used as a nanowire (or molecular wire) or part of a nanowire or a nanostructure to bridge a nanogap comprising two electrodes, the distance between which is in a range of 3 nm to 1 μm, preferably 5 nm to 100 nm, and most preferably 5 to 30 nm. The said nanostructure can be a nucleic acid dulex, a nucleic acid triplex, a nucleic acid quadruplex, a nucleic acid origami structure, or the combination thereof, or other nanostructures composed of nucleic acid bases or mixed nucleic acid bases and amino acid bases. The said electrodes comprise noble metals, for example, platinum (Pt), gold (Au), silver (Ag), palladium (Pd), rhodium (Rd), ruthenium (Ru), osmium (Os), and iridium (Ir), as well as other metals, such as copper (Cu), rhenium (Re), titanium (Ti), Niobium (Nb), Tantalum (Ta) and their derivatives, such as TiN, and TaN, etc., or their alloys. The two electrodes can form a nanogap by being placed next to each other on a non-conductive substrate or by being placed overlapping each other, separated by a non-conductive layer (ref. U.S. 62/890,251). An enzyme is attached to the nanowire or nanostructure for carrying out the biochemical reaction for the sensing, identification, or sequencing of biopolymers. The said biopolymers include but are not limited to DNA, RNA, DNA oligos, protein, peptides, polysaccharides, etc., either natural, modified, or synthesized. The enzymes include but are not limited to DNA polymerase, RNA polymerase, DNA helicase, DNA ligase, DNA exonuclease, reverse transcriptase, RNA primase, ribosome, sucrase, lactase, etc., either natural, mutated, or synthesized.

An embodiment of the invention is a system of a conductive or semiconductive molecular wire, comprising a nanostructure comprising one or more nucleic acid base pairs, wherein at least one nucleic acid base within the nanostructure is modified, and the presence of the modified nucleic acid base improves the conductance of the nanostructure in comparison to a canonical nucleic acid base in the same position.

A system for identification, characterization, or sequencing of a biopolymer comprising, a nanogap formed by a first electrode and a second electrode placed next to each other on a non-conductive substrate or placed overlapping each other separated by a non-conductive layer; a nanostructure comprising one or more nucleic acid base pairs that bridges the said nanogap by attaching one end to the first electrode and another end to the second electrode through a chemical bond, wherein at least one nucleic acid base within the nanostructure is modified, and the presence of the modified nucleic acid base improves the conductance of the nanostructure in comparison to a canonical nucleic acid base in the same position; a sensing probe attached to the nanostructure that can interact or perform a chemical or biochemical reaction with the biopolymer. further comprising, a bias voltage that is applied between the first electrode and the second electrode; a device that records a current fluctuation through the nanostructure caused by the interaction between the sensing probe and the biopolymer; and a software for data analysis that identifies or characterizes the biopolymer or a subunit of the biopolymer. In a further embodiment, the nanostructure is selected from the group consisting of a nucleic acid duplex, a nucleic acid triplex, a nucleic acid quadruplex, a nucleic acid origami structure, and a combination thereof. In a further embodiment, the nucleic acid base modification reduces the energy gap between

HOMO and LUMO in comparison to a canonical nucleic acid base in the same position without modification. In a further embodiment, the nanostructure comprises, a modified uracil (U^{m}), wherein U^{m} is 5-an alkenyl-uracil; or a modified thymine (T^{m}), wherein T^{m} is a 5-alkenyl-thymine; or a modified adenine (A^{m}), wherein A^{m} is a 7-deaza-7-alkenyl-adenine or a 7-propenyl-7-deaza-adenine; or a modified guanine (G^{m}), wherein G^{m} is a 7-deaza-7-alkenyl-2′-guanine; or a modified cytosine (C^{m}), wherein C^{m} is a 5-alkenyl-cytosine; or a modified guanine for electrode attachment (G^{s}), wherein G^{s} is a 8-(3-mercaptopropynyl)-deoxyguanosine or a 7-deaza-7-(3-mercaptppropynyl)-2′-deoxyguanosine with a disulfide; or a base pair of U^{m} and A^{m}, or a base pair of T^{m} and A^{m}, or a base pair of G^{m} and C^{m}, or a combination thereof; or a combination of the above. In a further embodiment, the biopolymer is selected from the group consisting of a DNA, a RNA, a protein, a polypeptide, an oligonucleotide, a polysaccharide, and their analogues, either natural, synthesized, or modified. In a further embodiment, the sensing probe is selected from the group consisting of a nucleic acid probe, a molecular tweezers, an enzyme, a receptor, a ligand, an antigen and an antibody, either native, mutated, expressed, or synthesized, and a combination thereof. In a further embodiment, the enzyme is selected from the group consisting of a DNA polymerase, a RNA polymerase, a DNA helicase, a DNA ligase, a DNA exonuclease, a reverse transcriptase, a RNA primase, a ribosome, a sucrase, laactase, either natural, mutated or synthesized. In a further embodiment, the nanogap size or the distance between the two electrodes is about 3 to 1000 nm, preferably about 5 to 100 nm, and most preferably about 5 to 30 nm. In a further embodiment, the electrodes are made using a noble metal selected from the group consisting of platinum (Pt), gold (Au), silver (Ag), palladium (Pd), rhodium (Rd), ruthenium (Ru), osmium (Os), iridium (Ir), or another metal selected from a group consisting of copper (Cu), rhenium (Re), titanium (Ti), Niobium (Nb), Tantalum (Ta) and their derivatives, such as TiN, and TaN or an alloy, and a combination thereof.

An embodiment is directed to a method for improving the conductance of a molecular wire, comprising, modifying at least one nucleic acid base so that the presence of the modified nucleic acid base improves the conductance of the molecular wire in comparison to a canonical nucleic acid base in the same position, wherein the molecular wire is a nanostructure comprising one or more nucleic base pairs.

Another embodiment is directed to a method for identification, characterization, or sequencing of a biopolymer comprising, forming a nanogap by placing a first electrode and a second electrode next to each other on a non-conductive substrate or overlapping each other separated by a non-conductive layer; providing a nanostructure comprising one or more nucleic acid base pairs with length comparable to the nanogap, wherein at least one nucleic acid base within the nanostructure is modified, and the presence of the modified nucleic acid base improves the conductance of the nanostructure in comparison to a canonical nucleic acid base at the same position; attaching one end of the nanostructure to the first electrode and another end to the second electrode through a chemical bond; and attaching a sensing probe to the nanostructure that can interact or perform a chemical or a biochemical reaction with the biopolymer. In a further embodiment, the embodiment further comprises applying a bias voltage between the first electrode and the second electrode; providing a device that records a current fluctuation through the nanostructure caused by the interaction between the sensing probe and the biopolymer; and providing a software for data analysis that identifies or characterizes the biopolymer or a subunit of the biopolymer. In a further embodiment, the nanostructure is selected from the group consisting of a nucleic acid duplex, a nucleic acid triplex, a nucleic acid quadruplex, a nucleic acid origami structure, and a combination thereof. In a further embodiment, the nucleic acid base modification reduces the energy gap between HOMO and LUMO in comparison to the canonical nucleic acid base in the same position without modification. In a further embodiment, the nanostructure comprises, a modified uracil (U^{m}), wherein U^{m} is a 5-alkenyl-uracil, or a modified thymine (T^{m}), wherein T^{m} is a 5-alkenyl-thymine; or a modified adenine (A^{m}), wherein A^{m} is a 7-deaza-7-alkenyl-adenine or a 7-propenyl deaza-adenine; or a modified guanine (G^{m}), wherein G^{m} is a 7-deaza-7-alkenyl-2′-guanine; or a modified cytosine (C^{m}), wherein C^{m} is a 5-alkenyl-cytosine; or a modified guanine for electrode attachment (G^{s}), wherein G^{s} is a 8-(3-mercaptopropynyl)-deoxyguanosine or a 7-deaza-7-(3-mercaptppropynyl)-2′-deoxyguanosine with a disulfide; or a base pair of U^{m} and A^{m}, or a base pair of T^{m} and A^{m}, or a base pair of G^{m} and C^{m}, or the combination thereof; or a combination of the above. In a further embodiment, the biopolymer is selected from the group consisting of a DNA, a RNA, a protein, a polypeptide, an oligonucleotide, a polysaccharide, and their analogies, either natural, synthesized, or modified. In a further embodiment, the sensing probe is selected from the group consisting of a nucleic acid probe, a molecular tweezers, an enzyme, a receptor, ligands, an antigen and an antibody, either native, mutated, expressed, or synthesized, and a combination thereof. In a further embodiment, the enzyme is selected from the group consisting of a DNA polymerase, a RNA polymerase, a DNA helicase, a DNA ligase, a DNA exonuclease, a reverse transcriptase, a RNA primase, a ribosome, a sucrase, a lactase, either natural, mutated or synthesized. In a further embodiment, the nanogap size or the distance between the two electrodes is about 3 to 1000 nm, preferably about 5 to 100 nm, and most preferably about 5 to 30 nm. In a further embodiment, the electrodes are made using a noble metal selected from a group consisting of platinum (Pt), gold (Au), silver (Ag), palladium (Pd), rhodium (Rd), ruthenium (Ru), osmium (Os), and iridium (Ir), or another metal selected from a group consisting of copper (Cu), rhenium (Re), titanium (Ti), Niobium (Nb), Tantalum (Ta) and their derivatives, such as TiN, and TaN or an alloy, and a combination thereof. In a further embodiment, the disclosure is directed to providing one or more nucleoside triphosphates selected from the group consisting of a 5-alkenyl-2′-deoxycytidine triphosphate, a 5-alkenyl-2′-deoxyuridine, a triphosphate, a 5-alkenyl-2′-deoxythymidine triphosphate, a 7-deaza-7-alkenyl-2′-deoxyadenosine triphosphate, a 7-deaza-7-alkenyl-2′-deoxyguanosine triphosphate, a 8-(3-mercaptopropynyl)-deoxyguanosine triphosphate, and a combination thereof; and incorporating the modified nucleic acid base into a nucleic acid strand within the nanostructure enzymatically using the nucleoside triphosphates provided.

General Remarks:

This invention describes the modification of nucleic bases for DNA engineering. The same principles or concepts and procedures apply to RNA engineering too.

All publications, patents, and other documents mentioned herein are incorporated by reference in their entirety.

Unless defined otherwise, all technical and scientific terms used herein have the same meaning as those commonly understood by one of ordinary skill in the art to which this invention belongs. While the present invention has been illustrated by a description of various embodiments and while these embodiments have been described in considerable detail, it is not the intention of the applicants to restrict or in any way limit the scope of the applications. Additional advantages and modifications will readily appear to those skilled in the art. The invention in its broader aspects is therefore not limited to the specific details, representative device, apparatus and method, and illustrative example shown and described. Accordingly, departures may be made from such details without departing from the spirit of applicant's general inventive concept.

### CITATIONS

1 Hasan, R. M. M. & Luo, X. Promising Lithography Techniques for Next-Generation Logic Devices. Nanomanufacturing and Metrology 1, 67-81 (2018).
2 Flood, A. H., Stoddart, J. F., Steuerman, D. W. & Heath, J. R. Whence Molecular Electronics? Science 306, 2055-2056 (2004).
3 Braun, E., Eichen, Y., Sivan, U. & Ben-Yoseph, G. DNA-templated assembly and electrode attachment of a conducting silver wire. Nature 391, 775-778, (1998).
4 Porath, D., Bezryadin, A., de Vries, S. & Dekker, C. Direct measurement of electrical transport through DNA molecules. Nature 403, 635-638, (2000).
5 de Pablo, P. J. et al. Absence of dc-Conductivity in □-DNA. Physical Review Letters 85, 4992-4995, doi:10.1103/PhysRevLett.85.4992 (2000).
6 Storm, A. J., Noort, J. v., Vries, S. d. & Dekker, C. Insulating behavior for DNA molecules between nanoelectrodes at the 100 nm length scale. Applied Physics Letters 79, 3881-3883, (2001).
7 Hartzell, B. et al. Comparative current-voltage characteristics of nicked and repaired A-DNA. Applied Physics Letters 82, 4800-4802, (2003).
8 Yoo, K. H. et al. Electrical Conduction through Poly(dA)-Poly(dT) and Poly(dG)-Poly(dC) DNA Molecules. Physical Review Letters 87, 198102, doi:10.1103/PhysRevLett.87.198102 (2001).
9 Kasumov, A. Y. et al. Proximity-Induced Superconductivity in DNA. Science 291, 280-282, (2001).
10 Kasumov, A. Y., Klinov, D. V., Roche, P. E., Guéron, S. & Bouchiat, H. Thickness and low-temperature conductivity of DNA molecules. Applied Physics Letters 84, 1007-1009, (2004).
11 Taniguchi, M. & Kawai, T. DNA electronics. Physica E: Low-dimensional Systems and Nanostructures 33, 1-12, (2006).
12 Liu, C. et al. Engineering nanometre-scale coherence in soft matter. Nat Chem 8, 941-945, (2016).
13 Slinker, J. D., Muren, N. B., Renfrew, S. E. & Barton, J. K. DNA charge transport over 34 nm. Nature Chemistry 3, 228, (2011).
14 Xiang, L. et al. Gate-controlled conductance switching in DNA. Nat Commun 8, 14471, (2017).
15 Guo, C. et al. Molecular rectifier composed of DNA with high rectification ratio enabled by intercalation. NATURE CHEMISTRY 8, 484-490, doi:10.1038/nchem.2480 (2016).
16 Hihath, J., Xu, B., Zhang, P. & Tao, N. Study of single-nucleotide polymorphisms by means of electrical conductance measurements. Proceedings of the National Academy of Sciences of the United States of America 102, 16979-16983, (2005).
17 Xu, B., Zhang, P., Li, X. & Tao, N. Direct Conductance Measurement of Single DNA Molecules in Aqueous Solution. Nano Lett. 4, 1105-1108 (2004).
18 Gomez, E. F. & Steckl, A. J. in Green Materials for Electronics, (eds Mihai Irimia-Vladu, Eric D. Glowacki, Niyazi S. Sariciftci, & Siegfried Bauer) 191-230 (2018).
19 Bixon, M. et al. Long-range charge hopping in DNA. PNAS 96, 11713-11716 (1999).
20 Xiang, L. et al. Intermediate tunnelling-hopping regime in DNA charge transport. Nat Chem 7, 221-226, (2015).
21 Renaud, N., Berlin, Y. A., Lewis, F. D. & Ratner, M. A. Between Superexchange and Hopping: An Intermediate Charge-Transfer Mechanism in Poly(A)-Poly(T) DNA Hairpins. Journal of the American Chemical Society 135, 3953-3963, (2013).
22 Heim, T., Deresmes, D. & Vuillaume, D. Conductivity of DNA probed by conducting—atomic force microscopy: Effects of contact electrode, DNA structure, and surface interactions. Journal of Applied Physics 96, 2927-2936, (2004).
23 Jimenez-Monroy, K. L. et al. High Electronic Conductance through Double-Helix DNA Molecules with Fullerene Anchoring Groups. The journal of physical chemistry. A 121, 1182-1188, (2017).
24 Wang, Y.-H. et al. Tunneling Decay Constant of Alkanedicarboxylic Acids: Different Dependence on the Metal Electrodes between Air and Electrochemistry. The Journal of Physical Chemistry C 118, 18756-18761, (2014).
25 Ogino, M., Taya, Y. & Fujimoto, K. Detection of methylcytosine by DNA photoligation via hydrophobic interaction of the alkyl group. Organic & Biomolecular Chemistry 7, 3163-3167, (2009).
26 Hanawa, K., Matsumoto, Y., Saito, R. & Saito, Y. 8-substituted guanosine derivative having hydrogen-bonding substituent and oligonucleotide containing the same. Japan patent (2008).
27 Barthelemy, A.-L. et al. Facile Preparation of Vinyl S-Trifluoromethyl NH Aryl Sulfoximines. European Journal of Organic Chemistry 2018, 3764-3770, (2018).
28 Zhu, X.-F., Jr., H. J. W. & Scott, A. I. An Improved Transient Method for the Synthesis of N-Benzoylated Nucleosides. SYNTHETIC COMMUNICATIONS 33, 1233-1243 (2003).
29 Aradi, B., Hourahine, B. & Frauenheim, T. DFTB+, a Sparse Matrix-Based Implementation of the DFTB Method. The Journal of Physical Chemistry A 111, 5678-5684, (2007).
30 Elstner, M. et al. Self-consistent-charge density-functional tight-binding method for simulations of complex materials properties. Physical Review B 58, 7260-7268, (1998).
31 Pecchia, A. & Carlo, A. D. Atomistic theory of transport in organic and inorganic nanostructures. Reports on Progress in Physics 67, 1497-1561, doi:10.1088/0034-4885/67/8/r04 (2004).
32 Pecchia, A., Penazzi, G., Salvucci, L. & Di Carlo, A. Non-equilibrium Green's functions in density functional tight binding: method and applications. New Journal of Physics 10, 065022, (2008).
33 https://www.dftbplus.org/fileadmin/DFTBPLUS/public/dftbplus/latest/manual.pdf

1. A system of a conductive or semiconductive molecular wire, comprising a nanostructure comprising one or more nucleic acid base pairs, wherein at least one nucleic acid base within the nanostructure is modified, and the presence of the modified nucleic acid base improves the conductance of the nanostructure in comparison to a canonical nucleic acid base in the same position.

2. A system for identification, characterization, or sequencing of a biopolymer comprising,
a. a nanogap formed by a first electrode and a second electrode placed next to each other on a non-conductive substrate or placed overlapping each other separated by a non-conductive layer;
b. a nanostructure comprising one or more nucleic acid base pairs that bridges the said nanogap by attaching one end to the first electrode and another end to the second electrode through a chemical bond, wherein at least one nucleic acid base within the nanostructure is modified, and the presence of the modified nucleic acid base improves the conductance of the nanostructure in comparison to a canonical nucleic acid base in the same position; and
c. a sensing probe attached to the nanostructure that can interact or perform a chemical or biochemical reaction with the biopolymer.

3. The system of claim 2, further comprising,
a. a bias voltage that is applied between the first electrode and the second electrode;
b. a device that records a current fluctuation through the nanostructure caused by the interaction between the sensing probe and the biopolymer; and
c. a software for data analysis that identifies or characterizes the biopolymer or a subunit of the biopolymer.

4. The system of claim 2, wherein the nanostructure is selected from the group consisting of a nucleic acid duplex, a nucleic acid triplex, a nucleic acid quadruplex, a nucleic acid origami structure, and a combination thereof.

5. The system of claim 2, wherein the nucleic acid base modification reduces the energy gap between HOMO and LUMO in comparison to a canonical nucleic acid base in the same position without modification.

6. The system of claim 2, wherein the nanostructure comprises,
a. a modified uracil (U^{m}), wherein U^{m} is a 5-alkenyl-uracil; or
b. a modified thymine (T^{m}), wherein T^{m} is a 5-alkenyl-thymine; or
c. a modified adenine (A^{m}), wherein A^{m} is a 7-deaza-7-alkenyl-adenine or a 7-propenyl-7-deaza-adenine; or
d. a modified guanine (G^{m}), wherein G^{m} is a 7-deaza-7-alkenyl-2′-guanine; or
e. a modified cytosine (C^{m}), wherein C^{m} is a 5-alkenyl-cytosine; or
f. a modified guanine for electrode attachment (G^{s}), wherein G^{s} is an 8-(3-mercaptopropynyl)-deoxyguanosine or a 7-deaza-7-(3-mercaptppropynyl)-2′-deoxyguanosine with a disulfide; or
g. a base pair of U^{m} and A^{m}, or a base pair of T^{m} and A^{m}, or a base pair of G^{m} and C^{m}, or a combination thereof; or
h. a combination of the above.

7. The system of claim 2, wherein the biopolymer is selected from the group consisting of a DNA, a RNA, a protein, a polypeptide, an oligonucleotide, a polysaccharide, and their analogues, either natural, synthesized, or modified.

8. The system of claim 2, wherein the sensing probe is selected from the group consisting of a nucleic acid probe, a molecular tweezers, an enzyme, a receptor, a ligand, an antigen and an antibody, either native, mutated, expressed, or synthesized, and a combination thereof.

9. The system of claim 8, wherein the enzyme is selected from the group consisting of a DNA polymerase, a RNA polymerase, a DNA helicase, a DNA ligase, a DNA exonuclease, a reverse transcriptase, a RNA primase, a ribosome, a sucrase, lactase, either natural, mutated or synthesized.

10. The system of claim 2, wherein the nanogap size or the distance between the two electrodes is about 3 to 1000 nm, preferably about 5 to 100 nm, and most preferably about 5 to 30 nm.

11. The system of claim 2, wherein the electrodes are made using a noble metal selected from the group consisting of platinum (Pt), gold (Au), silver (Ag), palladium (Pd), rhodium (Rd), ruthenium (Ru), osmium (Os), iridium (Ir), or another metal selected from a group consisting of copper (Cu), rhenium (Re), titanium (Ti), Niobium (Nb), Tantalum (Ta) and their derivatives, such as TiN, and TaN or an alloy, and a combination thereof.

12. A method for improving the conductance of a molecular wire, comprising, modifying at least one nucleic acid base so that the presence of the modified nucleic acid base improves the conductance of the molecular wire in comparison to a canonical nucleic acid base in the same position, wherein the molecular wire is a nanostructure comprising one or more nucleic base pairs.

13. A method for identification, characterization, or sequencing of a biopolymer comprising,
a. forming a nanogap by placing a first electrode and a second electrode next to each other on a non-conductive substrate or overlapping each other separated by a non-conductive layer;
b. providing a nanostructure comprising one or more nucleic acid base pairs with length comparable to the nanogap, wherein at least one nucleic acid base within the nanostructure is modified, and the presence of the modified nucleic acid base improves the conductance of the nanostructure in comparison to a canonical nucleic acid base at the same position;
c. attaching one end of the nanostructure to the first electrode and another end to the second electrode through a chemical bond; and
d. attaching a sensing probe to the nanostructure that can interact or perform a chemical or a biochemical reaction with the biopolymer.

14. The method of claim 13, further comprising,
a. applying a bias voltage between the first electrode and the second electrode;
b. providing a device that records a current fluctuation through the nanostructure caused by the interaction between the sensing probe and the biopolymer; and
c. providing a software for data analysis that identifies or characterizes the biopolymer or a subunit of the biopolymer.

15. The method of claim 13, wherein the nanostructure is selected from the group consisting of a nucleic acid duplex, a nucleic acid triplex, a nucleic acid quadruplex, a nucleic acid origami structure, and a combination thereof.

16. The method of claim 13, wherein the nucleic acid base modification reduces the energy gap between HOMO and LUMO in comparison to the canonical nucleic acid base in the same position without modification.

17. The method of claim 13, wherein the nanostructure comprises,
a. a modified uracil (U^{m}), wherein U^{m} is a 5-alkenyl-uracil, or
b. a modified thymine (T^{m}), wherein T^{m} is a 5-alkenyl-thymine; or
c. a modified adenine (A^{m}), wherein A^{m} is a 7-deaza-7-alkenyl-adenine or a 7-propenyl-7-deaza-adenine; or
d. a modified guanine (G^{m}), wherein G^{m} is a 7-deaza-7-alkenyl-2′-guanine; or
e. a modified cytosine (C^{m}), wherein C^{m} is a 5-alkenyl-cytosine; or
f. a modified guanine for electrode attachment (G^{s}), wherein G^{s} is an 8-(3-mercaptopropynyl)-deoxyguanosine or a 7-deaza-7-(3-mercaptppropynyl)-2′-deoxyguanosine with a disulfide; or
g. a base pair of U^{m} and A^{m}, or a base pair of T^{m} and A^{m}, or a base pair of G^{m} and C^{m}, or the combination thereof; or
h. a combination of the above.

18. The method of claim 13, wherein the biopolymer is selected from the group consisting of a DNA, a RNA, a protein, a polypeptide, an oligonucleotide, a polysaccharide, and their analogies, either natural, synthesized, or modified.

19. The method of claim 13, wherein the sensing probe is selected from the group consisting of a nucleic acid probe, a molecular tweezers, an enzyme, a receptor, ligands, an antigen and an antibody, either native, mutated, expressed, or synthesized, and a combination thereof.

20. The method of claim 19, wherein the enzyme is selected from the group consisting of a DNA polymerase, a RNA polymerase, a DNA helicase, a DNA ligase, a DNA exonuclease, a reverse transcriptase, a RNA primase, a ribosome, a sucrase, a lactase, either natural, mutated or synthesized.

21. The method of claim 13, wherein the nanogap size or the distance between the two electrodes is about 3 to 1000 nm, preferably about 5 to 100 nm, and most preferably about 5 to 30 nm.

22. The method of claim 13, wherein the electrodes are made using a noble metal selected from a group consisting of platinum (Pt), gold (Au), silver (Ag), palladium (Pd), rhodium (Rd), ruthenium (Ru), osmium (Os), and iridium (Ir), or another metal selected from a group consisting of copper (Cu), rhenium (Re), titanium (Ti), Niobium (Nb), Tantalum (Ta) and their derivatives, such as TiN, and TaN or an alloy, and a combination thereof.

23. The method of claim 13, further comprising,
a. providing one or more nucleoside triphosphates selected from the group consisting of a 5-alkenyl-2′-deoxycytidine triphosphate, a 5-alkenyl-2′-deoxyuridine triphosphate, a 5-alkenyl-2′-deoxythymidine triphosphate, a 7-deaza-7-alkenyl-2′-deoxyadenosine triphosphate, a 7-deaza-7-alkenyl-2′-deoxyguanosine triphosphate, an 8-(3-mercaptopropynyl)-deoxyguanosine triphosphate, and a combination thereof; and
b. incorporating the modified nucleic acid base into a nucleic acid strand within the nanostructure enzymatically using the nucleoside triphosphates provided.
